(19) United States
(12) Patent Application Publication
(10) Pub. No.: US 2026/0018257 A1
(43) Pub. Date: Jan. 15, 2026

(54) Method for Classifying Physical, Chemical and/or Physiological Properties of Molecules

(71) Applicant: Fraunhofer-Gesellschaft zur Förderung der angewandten Forschung e.V., München (DE)

(72) Inventors: Thilo Bauer, Forchheim (DE); Doris Schicker, Unterschleissheim (DE)

(21) Appl. No.: 18/993,712

(22) PCT Filed: Jul. 13, 2023
(86) PCT No.: PCT/EP2023/069455

§ 371 Date: Jan. 13, 2025

(30) Foreign Application Priority Data
Jul. 13, 2022 (DE) 10 2022 117 408.5

(51) Int. Cl. G16C 20/30; G16C 20/70

(52) U.S. Cl. CPC G16C 20/30; G16C 20/70

(57) ABSTRACT

A method for selecting molecules with a sought-after physical, chemical and/or physiological property from a group of molecules is provided, wherein a classification according to a chemical, physical and/or physiological property of a molecule is undertaken with the aid of a mathematical model. As a result, molecules with the sought-after property can be selected from the group of molecules. Subsequently, an experimental confirmation as to whether the molecules actually have the sought-after the physical, chemical and/or physiological property is undertaken for this selection of molecules. Also the method can be used to select at least one molecule with a sought-after chemical, physical and/or physiological property from a group of molecules and for identifying the influence of structure patterns in molecules on at least one chemical, physical and/or physiological property of molecules.

### CROSS-REFERENCE TO RELATED APPLICATIONS

This application is the United States national phase of International Patent Application No. PCT/EP2023/069455 filed Jul. 13, 2023, and claims priority to German Patent Application No. 10 2022 117 408.5 filed Jul. 13, 2022, the disclosures of each of which are hereby incorporated by reference in their entireties.

### BACKGROUND

### Technical Field

The present disclosure relates to a method for selecting molecules with a sought-after physical, chemical, and/or physiological property from a group of molecules, wherein a classification according to a chemical, physical, and/or physiological property of a molecule is undertaken with the aid of a mathematical model. As a result, molecules with the sought-after property can be selected from the group of molecules. For this selection of molecules, experimental confirmation is then undertaken to determine whether the molecules actually exhibit sought-after the physical, chemical, and/or physiological property. Furthermore, the use of the method according to the present disclosure for selecting at least one molecule with a sought-after chemical, physical, and/or physiological property from a group of molecules and for identifying the influence of structure patterns in molecules on at least one chemical, physical, and/or physiological property of molecules is described.

### Technical Considerations

Molecules have chemical, physical, and physiological properties. While physical properties can be quantified by measuring underlying physical characteristics, chemical properties can be quantified by measuring an underlying chemical characteristic in the reaction of a molecule with another substance. The physical properties of a molecule comprise, for example, the color of the molecule. Water solubility, on the other hand, is one of the chemical properties of a molecule.

Furthermore, molecules exhibit physiological properties. This comprises physical and chemical properties of substances from the perspective of their perceptibility or impact on the environment. Examples of this are the smell and taste of a molecule.

Chemical, physical, and physiological properties are of great interest for a wide range of applications. Physiological properties describe properties of molecules that have effects on the lives of organisms. According to the present disclosure, this comprises properties such as taste or smell of molecules. Furthermore, according to the present disclosure, this also comprises the grouping of molecules into permitted and non-permitted chemicals in cosmetics and personal care. This is regulated by the use authorization according to Articles Regulation, Annex II—Restricted Substances the Annex II of the European Chemicals Agency (ECHA). The taste of molecules directly appeals to the human sense of taste and thus has a decisive influence on human eating behavior and for example which foods are perceived as pleasant or unpleasant. The taste evoked by molecules is therefore of great importance, especially in the food industry.

Smell is one of the five human senses and plays an important role in daily life. For example, the smell of food influences our eating behavior [1], and smells in threatening situations influence the human memory of such situations [2]. In addition to the importance of smells for humans, they also play an important role in the economy, especially in the food and cosmetics industries, where the development of new flavors and the identification of odor-active molecules are essential. When developing new aromas, a predictive approach during molecular design is required, to reduce the space of candidate molecules from virtually all to a promising set of structures.

Unfortunately, although many advances have been made in odor prediction in recent years [3, 4, 5, 6], little is known about the relationship between the structure of a molecule and its odor, so that chemists cannot be provided with a “toolbox” to design molecular structures with a specific odor in mind [7, 8]. Furthermore, there is disagreement about the dimensionality of the olfactory space [9, 10]. To derive the rather vague property of odor from objectively measurable or calculable molecular properties, a relationship between physicochemical parameters and odor can be used. Using this approach and principal component analysis (PCA), Khan et al. predicted the pleasantness of the odor of molecules and identified it as one of the dimensions of human olfactory perception [11], in agreement with other studies [12].

To predict a specific odor, Keller et al. investigated the performance of 22 different machine learning models in predicting 19 odor descriptors. They used physiochemical properties such as the type of atoms, functional groups, or topological and geometric information. The models successfully predicted eight of the 19 descriptors considered. The authors looked for correlations between features and descriptors and found significant correlations between sulfur-containing molecules and the descriptors “garlic” and “burnt.” Based upon the good performance of the linear models, the authors concluded that there is a linear, summative effect of the features on odor perception [13], [14].

Shang et al. investigated different combinations of feature generation models and machine learning algorithms to predict the odor of molecules from ten possible descriptors. They applied the models in GC/O (gas chromatography analysis with olfactometric detection). With an accuracy of 97.08%, the Support Vector Machine (SVM) achieved the best results in the previous feature selection using Boruta [15]. However, when aroma molecules that were not included in the model building were predicted, the accuracy dropped to 70% [6]. The models used features calculated using the chemoinformatics software Dragon for odor prediction. These features are also used by Snitz et al. to predict the odor of odorant mixtures [5]. The training of a deep autoencoder [16] also enabled the extraction of features that can be used alternatively to using features generated by Dragon. Tran et al. developed the autoencoder DeepNose to extract molecular features. DeepNose features performed equally well in predicting odor perceptions, compared to Dragon features [3].

Although the models used are promising and useful in their own right, they use a variety of different features that do not provide deep insight into the mechanism of prediction. Due to their opaque nature, the prior-art models function more as a “black box,” whereby knowledge about the structure/odor relationships is still lacking.

This means that, in business and science, sensory-trained experts have to smell molecules in order to determine their odor. Due to largely unknown structure/odor relationships, the trial-and-error principle prevails in the development of flavorings or the identification of odor-active molecules. This is very time-consuming, requires a lot of personnel, and is therefore uneconomical.

It is equally desirable to be able to derive other physical, chemical, or physiological properties of a molecule from its structure.

Based upon the deficiencies in the prior art, it is therefore an object of the present disclosure to provide a method by which molecules with a desired physical, chemical, or physiological property can be selected from a given set of molecules without having to examine all molecules with regard to the desired property using experimental methods.

### SUMMARY

For this purpose, in some non-limiting embodiments, the present disclosure provides a method for selecting molecules with a desired physical, chemical, and/or physiological property from a group of molecules, comprising the steps of:
providing a group of Oₖ molecules, by a user, wherein k∈N;
providing a classification according to a chemical, physical, and/or physiological property of a molecule, having Cᵢ classes, wherein i∈N;
providing a mathematical model for the classification, wherein the mathematical model describes relationships G_{i,j} between a structure pattern and a class, by probabilities that a structure pattern Fⱼ of a molecule belongs to a class Cᵢ or a molecule of a class Cᵢ has a structure pattern Fⱼ,
selecting a weighting function aᵢⱼ for the mathematical model, by a user;
assigning all Oₖ molecules into the Cᵢ classes of the classification by the mathematical model, wherein the mathematical model comprises the steps of:
   a) determining and storing Fⱼ structure patterns of the chemical structure of each of the Oₖ molecules, with assignment to the corresponding molecule, wherein j∈N;
   b) assigning the probability G_{i,j} to each structure pattern Fⱼ of a molecule for each class Cᵢ and calculating the influence I_{i,j} according to the formula

$${I}_{i , j} = {a}_{i , j} \cdot {G}_{i , j}$$

for each structure pattern Fⱼ of one molecule for each class Cᵢ;
c) calculating a point value P_{i,k} for each molecule Oₖ, using

$${P}_{i , k} = {\sum }_{{F}_{j} ∈ {O}_{k}} {I}_{i , j}$$

for each class Cᵢ, wherein the influences I_{i,j} of all structure patterns Fⱼ comprised in a molecule Oₖ are summed for each class Cᵢ;
d) assigning each molecule to the class Cᵢ with the highest point value P_{i,k} for the corresponding molecule;
displaying and/or outputting the molecules with assignment to the classes of the classification, and optionally the associated point values P_{i,k}, the associated influences I_{i,j}, and the structure pattern Fⱼ;
selecting the molecules which have been assigned to the class with the sought-after physical, chemical, and/or physiological property;
confirming experimentally the physical, chemical, and/or physiological property of at least a portion of the selected molecules by a user; and/or verifying and/or identifying the relationship between at least one structure pattern Fⱼ and a class Cᵢ by a user.

In some non-limiting embodiments, the present disclosure also relates to the use of the method according to the present disclosure for selecting at least one molecule with a sought-after chemical, physical, and/or physiological property from a group of molecules and for identifying the influence of structure patterns in molecules on at least one chemical, physical, and/or physiological property of molecules.

### BRIEF DESCRIPTION OF THE DRAWINGS

The present disclosure is explained in more detail below with reference to two figures and three exemplary embodiments.

FIG. 1 shows a sequence of a non-limiting example of the method according to the present disclosure;

FIG. 2 shows results of a non-limiting example of the method according to the present disclosure.

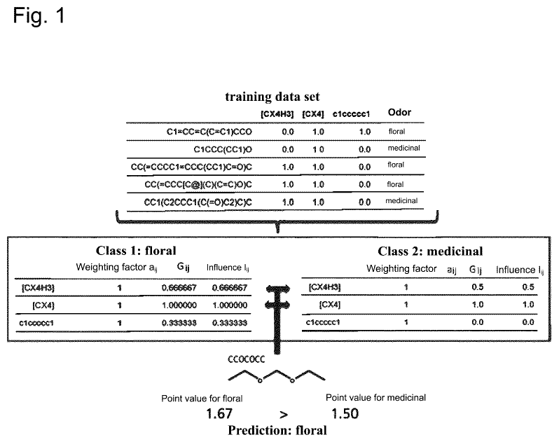

FIG. 1 represents a sequence of a non-limiting example of the method according to the present disclosure, which is described in more detail in exemplary embodiment 2.

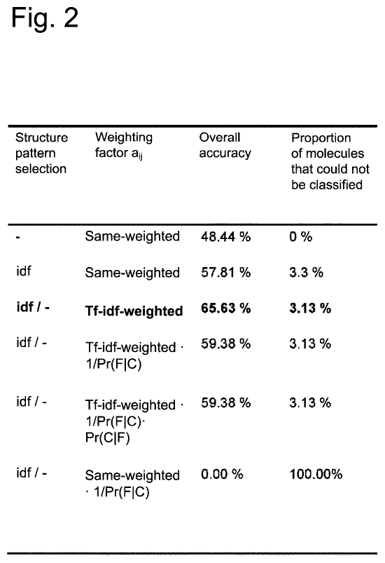

FIG. 2 represents results of a non-limiting example of the method according to the present disclosure, in which a mathematical model with different weighting functions aᵢⱼ and with and without selection of the structure patterns was implemented.

### DETAILED DESCRIPTION

According to some non-limiting embodiments of the present disclosure, a group of Oₖ molecules is provided by a user, wherein k∈N. In some non-limiting embodiments of the present disclosure, 20 to 1,000 molecules are provided, preferably 20 to 800 molecules are provided, and more preferably 20 to 300 molecules are provided. In this case, “provided” means first of all that the structural formulas of the molecules are available and are thus provided. This is possible, for example, by providing the molecules in the structural code SMILES, which encodes structure patterns as SMARTS [17, 18, 19]. In addition, however, it is possible in some non-limiting embodiments to have each of the molecules available as a substance at a later date for experimental confirmation.

According to some non-limiting embodiments of the present disclosure, there is a classification according to a chemical, physical, and/or physiological property of a molecule having Cᵢ classes, wherein i∈N.

In some non-limiting embodiments of the present disclosure, the classification is selected from structure-based properties of molecules, for example from the group comprising odor, taste, color, toxicity, water solubility, and/or permitted chemicals and/or non-permitted chemicals in cosmetics and/or personal care. In a preferred and non-limiting embodiment, the classification is a classification according to the odor of the molecules.

A classification comprises multiple classes; for example, the water solubility classification comprises the classes hydrophilic and hydrophobic. The toxicity classification comprises the classes toxic and non-toxic. The color classification can comprise different colors as classes—for example, blue, red, yellow, and/or green. The taste classification comprises different tastes, such as bitter, sour, sweet, salty, and umami. The odor classification comprises odor varieties such as ‘woody, resinous,’ ‘floral,’ ‘fruity, not lemony,’ ‘medicinal,’ ‘perfumed,’ ‘light,’ ‘heavy,’ ‘sweet,’ ‘aromatic,’ ‘fragrant,’ and/or ‘repugnant’ as classes. Preferably, the odor classification comprises the odor varieties ‘woody, resinous,’ ‘floral,’ ‘fruity, not-lemony,’ ‘medicinal,’ and/or ‘perfumed’ as classes.

Furthermore, there is a mathematical model for the classification, provided by a user. According to the disclosure, the mathematical model has probabilities G_{i,j} that a structure pattern Fⱼ of a molecule belongs to a class Cᵢ, or a molecule of a class Cᵢ has a structure pattern Fⱼ. The mathematical model has been previously trained using a training data set for the selected classification. A training data set has Oₗ molecules for which the assignment to a class Cᵢ in the classification is known, wherein l, i∈N. In a further non-limiting embodiment, a molecule may be assigned to multiple classes Cᵢ. The creation of the mathematical model is explained later in the present disclosure.

According to some non-limiting embodiments of the present disclosure, a weighting function aᵢⱼ for the mathematical model is selected by a user. A suitable weighting function aᵢⱼ is selected from the group of statistical measures, such as tf-idf functions, normalization function, equally weighted function. tf and idf values are calculated using the training data set, and the formulas generally known to a person skilled in the art [26].

Afterwards, all Oₖ molecules Cᵢ are assigned to classes of the classification by the mathematical model. The mathematical model comprises the following steps:
a) determining and storing Fⱼ structure patterns of the chemical structure of each of the Oₖ molecules, with assignment to the corresponding molecule, wherein j∈N;
b) assigning the probability G_{i,j} to each structure pattern Fⱼ of a molecule for each class Cᵢ and calculating the influence I_{i,j} according to the formula

$${I}_{i , j} = {a}_{i , j} \cdot {G}_{i , j}$$

for each structure pattern Fⱼ of one molecule for each class Cᵢ;
c) calculating a point value P_{i,k} for each molecule Oₖ, using

$${P}_{i , k} = {\sum }_{{F}_{j} ∈ {O}_{k}} {I}_{i , j}$$

for each class Cᵢ, wherein the influences I_{i,j} of all structure patterns Fⱼ comprised in a molecule Oₖ are summed for each class Cᵢ;
d) assigning each molecule to the class Cᵢ with the highest point value P_{i,k} for the corresponding molecule.

In step a), structure patterns Fⱼ of the chemical structure of each of the Oₖ molecules are determined. These are stored together with assignments to the corresponding molecule. All structure patterns of the Oₖ molecules are determined in this step. Structure patterns that do not occur in the training data set are assigned an influence I_{i,j} of zero, and are therefore not taken into account in the method.

According to some non-limiting embodiments of the method according to the present disclosure, each structure pattern Fⱼ of a molecule for each class Cᵢ is assigned a probability G_{i,j}. The corresponding probability G_{i,j} is derived from the mathematical model for each structure pattern Fᵢ for a given classification. The influence I_{i,j} is calculated according to the formula

$${I}_{i , j} = {a}_{i , j} \cdot {G}_{i , j} \qquad ( 1 )$$

for each class Cᵢ. a_{i,j} corresponds to the previously selected weighting function. The weighting function can take into account additional information about the relationship between structure pattern and class, such as selectivity and specificity using the tf-idf function. A point value P_{i,k} is then calculated for each molecule Oₖ according to the formula

$${P}_{i , k} = {\sum }_{{F}_{j} ∈ {O}_{k}} {I}_{i , j} \qquad ( 2 )$$

for each class Cᵢ. According to the formula, the influences I_{i,j} of all structure patterns Fⱼ comprised in a molecule Oₖ are summed for each class Cᵢ. According to the present disclosure, for each class Cᵢ of the classification, a point value P_{i,k} is calculated for a molecule. The molecule is then assigned to the class Cᵢ of the classification that has the highest point value P_{i,k} for the corresponding molecule. According to the present disclosure, the molecule is assigned to at least one class Cᵢ. In some non-limiting embodiments of the present disclosure, the molecule is assigned to multiple classes. This happens if the highest point value P_{i,k} is the same for multiple classes. The assignment is then made to the classes Cᵢ for which the same highest point value P_{i,k} was determined.

In some non-limiting embodiments of the present disclosure, it is provided that, if the point values of a molecule are the same for all classes Cᵢ, this molecule be labeled as unpredictable. This can happen, for example, if a molecule consists entirely of structure patterns that do not occur in the training data set, and that therefore each have an influence I_{i,j} of zero.

The mathematical model therefore allows the molecules to be assigned to the classes Cᵢ of the classification. The mathematical model is based upon the assumption that each structure pattern has a certain influence on a class, and that a structure pattern/class relationship exists. The present disclosure thus enables sorting the Oₖ molecules into the classes of the classification. By applying the mathematical model, a pre-selection is made of molecules that are contained in the provided group of molecules and have the physical, chemical, or physiological property sought.

This allows a user to target a smaller selection of molecules of the Oₖ molecules for further experimental investigations, in order to find molecules with desired physical, chemical, and/or physiological properties. Advantageously, it is not necessary as before to subject all Oₖ molecules to experimental investigations. Preference can be given in experimental confirmation to the molecules with the highest point values in a class of the classification, and thus with a desired physical, chemical, and/or physiological property. Experimentally, it is confirmed whether a molecule actually has the physical, chemical, and/or physiological properties that it should have according to its classification.

For example, if molecules from a group that have the odor ‘floral’ are to be filtered out, the mathematical model for odor classification is applied, and the molecules that are assigned to the class ‘floral’ are then subjected to experimental confirmation. It is advantageous to start with the molecule that has the highest point value P_{i,k} in this class. Subsequently, further molecules in this class can be investigated experimentally, wherein these are advantageously arranged in a sequence according to descending point values P_{i,k} and investigated experimentally. In some non-limiting embodiments, only the molecule with the highest point value in a class is investigated experimentally. In a some non-limiting embodiments of the present disclosure, all molecules are experimentally investigated whose point value P_{i,k} deviates by at most 50%, preferably at most 30%, more preferably at most 10% from the highest point value P_{i,k} in this class. In some non-limiting embodiments of the present disclosure, all molecules of a class of the classification are investigated experimentally.

According to the present disclosure, the molecules Oₖ assigned to the classes of the classification are displayed and/or output. In some non-limiting embodiments, the molecules are displayed and/or output in such a way that the molecules are arranged in descending order according to their point value P_{i,k} in a class Cᵢ, starting with the molecule with the highest point value P_{i,k}. In some non-limiting embodiments, the associated point value P_{i,k} and/or the associated influences I_{i,j} and/or the associated structure pattern Fⱼ are displayed and/or output.

Subsequently, the molecules that have been assigned to the class with the desired physical, chemical, and/or physiological property are selected.

As already described, this is followed by experimental confirmation of the physical, chemical, and/or physiological properties of at least some of the selected molecules by a user. The experimental verification simultaneously checks the classification of the molecule by a user. The type of experimental confirmation depends upon the classification that was made. The following table provides a non-exhaustive overview of common experimental methods that can be used to investigate physical, chemical, and physiological properties of molecules. All other common experimental methods known to a person skilled in the art are equally applicable.

| Classification | Experimental confirmation |
|---|---|
| Taste | Taste test by trained person |
| Odor | Odor test by trained person |
| Water solubility | Conductivity measurements to determine the |
| | solubility product |
| Color | Spectroscopy |

In some non-limiting embodiments of the present disclosure, a verification and/or identification of the relationship between at least one structure pattern Fⱼ and a class Cᵢ is undertaken by a user. This advantageously makes it possible to gain insight into the structure pattern/class relationship. Physical, chemical, and/or physiological properties of molecules can thus be traced back to certain structure patterns of the molecules.

The present disclosure thus enables significant savings in personnel and technical effort, since it is no longer necessary to experimentally investigate all molecules Oₖ of a group in order to select at least one molecule of a certain class—and thus having a certain physical, chemical, and/or physiological property. By applying the mathematical model, a selection of molecules is made, and the subsequent experimental confirmation can be carried out specifically with this selection of molecules. This saves time and money compared to methods of the prior art. In addition, it is not necessary to have all molecules available as substances for experimental investigations, which saves upon additional costs.

According to some non-limiting embodiments, of the present disclosure, a mathematical model is used which comprises the probability G_{i,j} for defined structure patterns for defined classes Cᵢ of a classification, or a molecule of a class Cᵢ which has a structure pattern Fⱼ.

For this purpose, the mathematical model is trained according to the present disclosure by means of a training data set for a selected classification, wherein a training data set having Oₗ molecules of known class Cᵢ is specified, wherein l, i∈N. In this context, learning means nothing other than the probabilities Gᵢⱼ=PR(Fⱼ|Cᵢ) for defined structure patterns for defined classes Cᵢ being calculated using a given data set, or the probabilities Gᵢⱼ=PR(Cⱼ|Fᵢ) that a molecule of a class Cᵢ has a structure pattern Fⱼ. The structure patterns of the molecules in the data set are known, as well as the class in which the corresponding molecules belong. In some non-limiting embodiments of the present disclosure, a molecule may also be assigned to multiple classes.

In some non-limiting embodiments, the procedure for training the mathematical model comprises the following steps:
i. determining and storing Fⱼ structure patterns of the chemical structure of each molecule, with assignment to the corresponding molecule j∈N;
ii. Calculating the probability G_{i,j} that a structure pattern Fⱼ belongs to a class Cᵢ, wherein

$${G}_{ij} = Pr ( {F}_{j} \vert {C}_{i} ) = \frac{Pr ( {F}_{j} ⋂ {C}_{i} )}{Pr ( {C}_{i} )}$$

or
calculating the probability G_{i,j} that a molecule of a class Cᵢ has a structure pattern Fⱼ, wherein

$${G}_{i j} = Pr ( {C}_{i} \vert {F}_{j} ) = \frac{Pr ( {F}_{j} ⋂ {C}_{i} )}{Pr ( {F}_{j} )} .$$

In step i., the structure patterns Fⱼ of each molecule are determined. A structure pattern is a partial fragment of the chemical structure of the molecule. It is not necessary to use all structural components of the molecules. Rather, a prior feature selection can be carried out using an algorithm or statistical values.

For example, the determination of the structure pattern Fⱼ of a molecule can be made using so-called fingerprint algorithms. One known fingerprint algorithm from the prior art is the RDKit topology fingerprint [20, 21]. Furthermore, Dragon software [22] and graph convolutional neural networks [23] are known for determining molecular structures. A new method considers molecules as graphs and converts nodes and edges of the graphs into a vector, which allows molecules to be represented purely based upon structure [24].

In some non-limiting embodiments of the present disclosure, not all structure patterns occurring in a group of molecules are used in the method according to the present disclosure. In this case, the Fⱼ structure patterns which are determined and stored in method step a) according to the present disclosure constitute a selection from a larger number of structure patterns. The selection can be made, for example, by an algorithm, an idf weighting, or a tf-idf weighting. For example, an algorithm can make a selection based upon the minimum number of molecules that exhibit a structure pattern or based upon correlations between different structure patterns.

For each structure pattern Fⱼ, a probability G_{i,j} that a structure pattern belongs to a class Cᵢ is then calculated. The probability G_{i,j} is calculated using the formula

$${G}_{ij} = Pr ( {F}_{j} \vert {C}_{i} ) = \frac{Pr ( {F}_{j} ⋂ {C}_{i} )}{Pr ( {C}_{i} )} . \qquad ( 3 )$$

Alternatively, for each structure pattern Fⱼ, a probability G_{i,j} is then calculated that a molecule of a class Cᵢ has a structure pattern Fⱼ. The probability G_{i,j} is calculated using the formula

$${G}_{i j} = Pr ( {C}_{i} \vert {F}_{j} ) = \frac{Pr ( {F}_{j} ⋂ {C}_{i} )}{Pr ( {F}_{j} )} . \qquad ( 4 )$$

The present disclosure can be used to select molecules having a desired chemical, physical, and/or physiological property from a group of molecules.

Furthermore, the present disclosure can be used to identify the influence of structure patterns in molecules on at least one chemical, physical, and/or physiological property of molecules.

In a preferred and non-limiting embodiment, the method according to the present disclosure is used to determine the odor of a molecule or to select from a group of molecules the molecules that have a certain odor. In this case, the classification is the odor, and the classes can be individual odors, such as ‘floral’ and/or ‘medicinal.’

Advantageously, the method according to the present disclosure also provides an insight into the structure pattern/odor relationship. Since the method calculates for each structure pattern an influence I_{i,j} in the form of a quantitative value for each class and thus for each odor, by comparing these influences, structure patterns can be identified which appear to have a strong effect on a particular odor. The structure patterns can therefore also be arranged according to their influence on a particular odor.

### Exemplary Embodiment 1—Odor Determination

A mathematical model was as an example trained using a group of 5 molecules to classify odors into two classes: ‘floral’ and ‘medicinal.’ This means that structure patterns were determined for all molecules Fⱼ. For each of the 5 molecules, the class membership(s) was known. With this information, the probabilities G_{i,j} for each structure pattern Fⱼ were calculated. FIG. 1 lists the 5 molecules for the training data set. The molecules are represented in the structural code SMILES; the structure patterns are coded as SMARTS. For the sake of clarity, the three structure patterns [CX4H3], [CX4], c1ccccc1 have been shown as examples. For each of the 5 molecules, the classification as ‘floral’ or ‘medicinal’ was known. Structure patterns with the value 1.0 in the table occur in the corresponding molecule, and structure patterns with the value 0.0 do not occur in the corresponding molecule.

From the training data set, the probabilities Gᵢⱼ for each of the three structure patterns for the class ‘floral’ and for the class ‘medicinal’ were calculated using formula (3).

From a group of 10 molecules, those that have a ‘floral’ odor should then be filtered out. The procedure according to the present disclosure is explained in more detail below using one of the 10 molecules as an example. For the molecule CCOCOCC, the structure patterns of the training data set which occur in this molecule were determined. Furthermore, the weighting function aᵢⱼ was set as an equal weighting, so that all weighting factors were 1. According to formula (1), the influences Iᵢⱼ were then calculated for all structure patterns. The results for both classes for all 3 structure patterns are shown in FIG. 1. The molecule CCOCOCC has only the structure patterns [CX4H3], [CX4], such that the influences of these structure patterns in both classes were summed according to formula (2). This resulted in a point value of P_{i,k}=1.67 for the class ‘floral’ and a point value of P_{i,k}=1.50 for the class ‘medicinal.’ The molecule was then assigned to the class ‘floral.’ All other 9 molecules were classified according to the same principle. Three molecules could be assigned to the class ‘floral’ and seven molecules to the class ‘medicinal.’ These 3 molecules were then selected.

Of the 3 molecules in the floral class, the molecule CCOCOCC had the highest point value. Due to the manageable number of molecules that were assigned to the class ‘floral,’ all three molecules were investigated experimentally below. Substances, each consisting of the 3 molecules, were examined by a person trained in the perception of odors, and it was found that all three molecules could indeed be assigned to the class ‘floral’ in the experimental confirmation.

### Exemplary Embodiment 2—Validation of Mathematical Model

The method according to the present disclosure was carried out on a group of 64 molecules. The 64 molecules were classified into the odor classes ‘floral,’ ‘medicinal,’ ‘woody, resinous,’ ‘repugnant,’ ‘fruity, non-lemony,’ and ‘perfumed.’ To train the model, 63 of the 64 molecules were used, wherein their class membership in each case was known. A mathematical model for odor classification was created. The class of the remaining molecule was then calculated using the mathematical model. For this purpose, different weighting functions aᵢⱼ and/or different selections of structure patterns were used. The following table in FIG. 2 presents the results. The accuracy when estimating the odor of a molecule is 21.35%. This means that the method according to the present disclosure can classify the odor of molecules with at least twice the accuracy than if it is only estimated. The results of the classification using the mathematical model were most accurate when aᵢⱼ was a tf-idf weighting. The accuracy in this case was over 65%. For calculating the accuracy, all molecules that could not be classified were counted as ‘incorrect.’

For two of the molecules, no classification could be calculated. In one case, hexanol showed only structure patterns that occur in all classes. For thiophene, which in turn has only structure patterns that occur exclusively in this one molecule of the 64 molecules, the mathematical model could therefore not provide probabilities for these structure patterns.

### Exemplary Embodiment 3—Authorization for Use of Chemicals in the Cosmetics Industry and Personal Care

The method according to the present disclosure was used to predict the use approval of chemicals in cosmetics and personal care. For this purpose, a dataset consisting of 800 molecules (400 with and 400 without use approval) and 500,047 structural fragments was used to train the mathematical model. The mathematical model with the tf-idf-weighted conditional probability Pr(Cⱼ|Fⱼ) was able to replicate with an accuracy of over 85% whether molecules in the training data set have use approval. For 200 additional molecules (100 with, 100 without use approval), the application prediction was made using the mathematical model. The results were compared with FCM and Articles Regulation, Annex II—Restricted Substances the Annex II of the European Chemicals Agency (ECHA). Only 11 molecules were incorrectly classified as allowed.

The methods and the mathematical model, as discussed herein, may comprise, be implemented by, and/or be performed by at least one computing device (e.g., at least one processor thereof). For example, a computing device may perform one or more of the methods described herein. As another example, at least one non-transitory computer-readable medium may comprise instructions that, when executed by at least one processor, cause the at least one processor to perform any of the methods described herein and/or to execute the mathematical model described herein. In some non-limiting embodiments, the at least one processor may be implemented in hardware, firmware, or a combination of hardware and software.

Overall, the accuracy was 81%. Thus, the method according to the present disclosure can significantly save upon labor and personnel costs in the synthesis of chemicals for cosmetics and personal care by focusing more on predicted permitted substances.

### LITERATURE

[1] a) L. G. Fine, C. E. Riera, Frontiers in physiology 2019, 10, 1151; b) P. Morquecho-Campos, K. de Graaf, S. Boesveldt, Food quality and preference 2020, 85, 103959.
[2] J. E. Taylor, H. Lau, B. Seymour, A. Nakae, H. Sumioka, M. Kawato, A. Koizumi, Frontiers in Neuroscience 2020, 14, 255.
[3] N. B. Tran, D. R. Kepple, S. A. Shuvaev, A. A. Koulakov, International Conference on Machine Learning 2019, 6305.
[4] a) A. Keller, R. C. Gerkin, Y. Guan, A. Dhurandhar, G. Turu, B. Szalai, J. D. Mainland, Y. Ihara, C. W. Yu, R. Wolfinger, Science 2017, 355, 820; b) H. Li, B. Panwar, G. S. Omenn, Y. Guan, Gigascience 2018, 7, gix127.
[5] K. Snitz, A. Yablonka, T. Weiss, I. Frumin, R. M. Khan, N. Sobel, PLoS computational biology 2013, 9, e1003184.
[6] L. Shang, C. Liu, Y. Tomiura, K. Hayashi, Analytical chemistry 2017, 89, 11999.
[7] a) C. S. Sell, Angewandte Chemie International Edition 2006, 45, 6254; b) M. Genva, T. Kenne Kemene, M. Deleu, L. Lins, M.-L. Fauconnier, International journal of molecular sciences 2019, 20, 3018.
[8] K. J. Rossiter, Chemical reviews 1996, 96, 3201.
[9] K. Kaeppler, F. Mueller, Chemical senses 2013, 38, 189.
[10] R. Kumar, R. Kaur, B. Auffarth, A. P. Bhondekar, PloS one 2015, 10, e0141263.
[11] R. M. Khan, C.-H. Luk, A. Flinker, A. Aggarwal, H. Lapid, R. Haddad, N. Sobel, Journal of Neuroscience 2007, 27, 10015.
[12] a) M. Zarzo, Journal of Sensory Studies 2008, 23, 354; b) A. Koulakov, B. E. Kolterman, A. Enikolopov, D. Rinberg, Frontiers in systems neuroscience 2011, 5, 65.
[13] M. B. Kursa, W. R. Rudnicki, J Stat Softw 2010, 36, 1.
[14] A. Keller, e-Neuroforum 2003, 9, 121.
[15] M. B. Kursa, A. Jankowski, W. R. Rudnicki, Fundamenta Informaticae 2010, 101, 271.
[16] G. E. Hinton, R. R. Salakhutdinov, Science 2006, 313, 504.
[17] D. Weininger, Journal of chemical information and computer sciences 1988, 28, 31.
[18] Daylight Chemical Information Systems, Inc., “3. SMILES—A Simplified Chemical Language,” can be found under https://www.daylight.com/dayhtml/doc/theory/theory.smiles.html, 2019,
[19] Daylight Chemical Information Systems, Inc., “4. SMARTS—A Language for Describing Molecular Patterns,” can be found under https://www.daylight.com/dayhtml/doc/theory/theory.smarts.html, 2019.
[20] https://doi.org/10.1186/s13321-020-00445-4
[21] https://www.rdkit.org/UGM/2012/Landrum_RDKit_UGM.Fingerprints. Final.pptx.pdf
[22] http://www.talete.mi.it/products/dragon_molecular_descriptor_list.pdf
[23] https://ai.googleblog.com/2019/10/learning-to-smell-using-deep-learning.html
[24] arXiv: 1910.10685v2
[25] Chris Manning and Hinrich Schütze, Foundations of Statistical Natural Language Processing, MIT Press. Cambridge, MA: May 1999
[26] Heiner Strickenschmidt, Ontologien: Konzepte, Technologien und Anwendungen, Springer Verlag, 2009

1. A method for selecting molecules with a sought-after physical, chemical, and/or physiological property from a group of molecules, comprising:
providing a group of Oₖ molecules, by a user, wherein k∈N;
providing a classification according to a chemical, physical, and/or physiological property of a molecule, having Cᵢ classes, wherein i∈N;
providing a mathematical model for the classification, wherein the mathematical model describes relationships G_{i,j} between a structure pattern and a class, by probabilities that a structure pattern Fⱼ of a molecule belongs to a class Cᵢ or a molecule of a class Cᵢ has a structure pattern Fⱼ;
selecting a weighting function aᵢⱼ for the mathematical model, by a user;
assigning all Oₖ molecules into the Cᵢ classes of the classification by the mathematical model, wherein the mathematical model:
a) determines and stores Fⱼ structure patterns of the chemical structure of each of the Oₖ molecules, with assignment to the corresponding molecule, wherein j∈N;
b) assigns the probability G_{i,j} to each structure pattern Fⱼ of a molecule for each class Cᵢ and calculates the influence I_{i,j} according to the formula
$${I}_{i , j} = {a}_{i , j} \cdot {G}_{i , j}$$
for each structure pattern Fⱼ of one molecule for each class Cᵢ;
c) calculates a point value P_{i,k} for each molecule Oₖ, using
$${P}_{i , k} = {\sum }_{{F}_{j} ∈ {O}_{k}} {I}_{i , j}$$
for each class Cᵢ, wherein the influences I_{i,j} of all structure patterns Fⱼ comprised in a molecule Oₖ are summed for each class Cᵢ; and
d) assigns each molecule to the class Cᵢ with the highest point value P_{i,k} for the corresponding molecule;
displaying and/or outputting the molecules with assignment to the classes of the classification, and optionally the associated point values P_{i,k}, the associated influences I_{i,j}, and the structure pattern Fⱼ;
selecting the molecules which have been assigned to the class with the sought-after physical, chemical, and/or physiological property;
confirming experimentally the physical, chemical, and/or physiological property of at least a portion of the selected molecules by a user; and/or verifying and/or identifying the relationship between at least one structure pattern Fⱼ and a class Cᵢ by a user.

2. The method according to claim 1, wherein the display and/or output of at least some of the molecules is carried out such that the molecules are arranged in descending order according to their point value P_{i,k} in a class Cᵢ, starting with the molecule with the highest point value P_{i,k}.

3. The method according to claim 1, wherein the mathematical model is trained by a training data set for the selected classification, wherein a training data set having Oₗ molecules of known class Cᵢ is specified, wherein l, i∈N, the method further comprising:
i. determining and storing Fⱼ structure patterns of the chemical structure of each molecule, with assignment to the corresponding molecule j∈N;
ii. calculating the probability G_{i,j} that a structure pattern Fⱼ belongs to a class Cᵢ, wherein
$${G}_{ij} = Pr ( {F}_{j} \vert {C}_{i} ) = \frac{Pr ( {F}_{j} ⋂ {C}_{i} )}{Pr ( {C}_{i} )} ,$$
or
calculating the probability G_{i,j} that a molecule of a class Cᵢ has as structure pattern Fⱼ, wherein
$${G}_{i j} = Pr ( {C}_{i} \vert {F}_{j} ) = \frac{Pr ( {F}_{j} ⋂ {C}_{i} )}{Pr ( {F}_{j} )} .$$

4. The method according to claim 3, wherein determining and storing the Fⱼ structure patterns comprises selecting, using an algorithm, an idf weighting, or a tf-idf weighting.

5. The method according to claim 1, wherein the classification is selected from a group of structure-based properties of molecules comprising smell, taste, color, water solubility, toxicity, permitted chemicals and/or non-permitted chemicals in cosmetics and/or personal care.

6. The method according to claim 1, wherein the weighting function aᵢⱼ is selected from the group of statistical measures comprising tf-idf functions, normalization function, or equally-weighted function.

7. The method according to claim 1, wherein all molecules are experimentally investigated which have a point value P_{i,k} which deviates by at most 50% from the highest point value P_{i,k} in this class.

8. At least one molecule with a sought-after chemical, physical, and/or physiological property selected from a group of molecules evaluated according to the method of claim 1.

9. A method for identifying the influence of at least one structure patterns on at least one chemical, physical, and/or physiological property of a group of molecules evaluated according to the method of claim 1.

10. The method according to claim 1, wherein all molecules are experimentally investigated which have a point value P_{i,k} which deviates by at most 30% from the highest point value P_{i,k} in this class.

11. The method according to claim 1, wherein all molecules are experimentally investigated which have a point value P_{i,k} which deviates by at most 10% from the highest point value P_{i,k} in this class.

12. The method according to claim 1, wherein the group of Oₖ molecules comprises 20 to 1,000 molecules.
